(19) United States
(12) Patent Application Publication
(10) Pub. No.: US 2026/0306750 A1
(43) Pub. Date: Oct. 8, 2026

(54) Dose Divider

(71) Applicant: SANOFI PASTEUR, INC., Swiftwater, PA (US)

(72) Inventors: Michael Thomas Hrynyk, Cambridge, MA (US); Qinglian Li, Cambridge, MA (US); Nausheen Rahman, Cambridge, MA (US)

(21) Appl. No.: 19/483,650

(22) PCT Filed: May 10, 2024
(86) PCT No.: PCT/US2024/028781

§ 371 Date: Nov. 12, 2025

(30) Foreign Application Priority Data
May 11, 2023 (EP) 23172952.6

(51) Int. Cl. A61M 5/315

(52) U.S. Cl. CPC A61M 5/31593; A61M 5/3153; A61M 2209/04

(57) ABSTRACT

A dose divider for releasable attachment to a plunger rod of a syringe comprises first and second arms. A front portion of the first arm and the front portion of the second arm form a channel therebetween for receiving the plunger rod. A resilient hinge pivotably couples the first arm and the second arm such that the dose divider is reversibly movable between first and second configurations. When the dose divider is in the second configuration, the dose divider is configured to receive the plunger rod in the channel through an opening formed between the front portions of the first and second arms. The channel has a shape configured to receive a specific outer shape of the plunger rod. The front portions of the first and second arms are closer when the dose divider is in the first configuration than when the dose divider is in the second configuration.

### TECHNICAL FIELD

The present disclosure relates to a dose divider for attachment to the plunger rod of a syringe, to a system comprising a dose divider and a syringe, and to a method of using a dose divider and a syringe.

### BACKGROUND

Respiratory syncytial virus (RSV) is an important cause of severe acute lower respiratory illness (LRI) in infants and children and a common cause of severe pneumonia requiring hospital admission in children worldwide. According to global estimates, RSV caused approximately 33 million cases of LRI and approximately 118,000 deaths in children <5 years of age in 2015. Greater than 80% of all RSV-associated LRIs (RSV-LRIs) and more than 50% of the RSV-associated deaths in low- and middle-income countries were estimated to occur in infants ≥6 months old.

Syringes may typically comprise a container of medicament having an outlet through which the medicament can be expelled from the container during medicament delivery, and a plunger rod operable by a user to cause the delivery of medicament from the outlet. Such plunger rods may, for example, comprise a plunger or piston within the medicament container which can be moved into the medicament container to dispense medicament.

Some medicaments are intended to be delivered in two or more doses from a single container. In such cases, a user may be required to manipulate the plunger rod over a first range of motion to dispense a first dose, and then manipulate the plunger rod over one or more subsequent range(s) of motion to dispense subsequent doses. Some patients may have difficulty in manipulating the plunger rod to accurately dispense the intended separate doses with the intended quantities of medicament in each dose. Alternatively, patients may accidentally dispense unintended quantities of medicament, or dispense all of the medicament at once, through inexperience with the device or misunderstanding of device usage instructions.

### SUMMARY

According to a first aspect of the present disclosure, there is provided a dose divider for releasable attachment to a plunger rod of a syringe, the dose divider comprising:
a first arm and a second arm opposite the first arm, each comprising a respective front portion, wherein the front portion of the first arm and the front portion of the second arm form a channel therebetween for receiving the plunger rod; and
a resilient hinge pivotably coupling the first arm and the second arm such that the dose divider is reversibly movable between a first configuration and a second configuration,
wherein, when the dose divider is in the second configuration, the dose divider is configured to receive the plunger rod in the channel through an opening formed between the front portion of the first arm and the front portion of the second arm, the channel having a shape configured to receive a specific outer shape of the plunger rod, and
wherein the front portion of the first arm and the front portion of the second arm are closer when the dose divider is in the first configuration than when the dose divider is in the second configuration. This may provide a dose divider that is easy to control/operate by a user. This may provide a dose divider that is easier to reposition on a plunger rod.

The resilient hinge may be configured to bias the dose divider towards the first configuration. This may provide a dose divider that securely grips a plunger rod and/or is adaptable to more than one different type of plunger rod.

The first arm and the second arm may each comprise a respective rear portion, wherein the dose divider may be configured to be moved from the first configuration to the second configuration by a user moving the rear portion of the first arm towards the rear portion of the second arm. This may provide a dose divider that is easy to control/operate by a user. This may provide a dose divider that is easier to reposition on a plunger rod.

An outer surface of the rear portion of the first arm and an outer surface of the rear portion of the second arm may comprise respective grip formations. This may provide a dose divider that is easy to control/operate by a user, for example a user with reduced dexterity.

Each grip formation may comprise a plurality of ridges. This may provide a dose divider that is easy to control/operate by a user, for example a user with reduced dexterity.

The dose divider may further comprise a stop formation located on at least one of the rear portion of the first arm and the rear portion of the second arm, wherein the stop formation may be located between the first arm and the second arm to limit movement of the rear portion of the first arm towards the rear portion of the second arm. This may provide a dose divider that is easy to control/operate by a user. This may prevent damage to the dose divider from the rear portions being moved too close together.

The stop formation may comprise a first protrusion located on the rear portion of the first arm and a second protrusion located on the rear portion of the second arm, wherein the first protrusion and the second protrusion may be configured to be brought into contact with each other as the rear portion of the first arm is moved towards the rear portion of the second arm, to limit further movement of the rear portion of the first arm towards the rear portion of the second arm.

The first protrusion may comprise a groove configured to receive at least a portion of the second portion when the first protrusion and the second protrusion are brought into contact with each other.

The front portion of the first arm and the front portion of the second arm may each comprise a respective guide surface for guiding the plunger rod through the opening towards the channel. This may provide a dose divider that is easy to control/operate by a user, making it easier to attach the dose divider to a plunger rod.

The dose divider may further comprise at least one of a first blocking projection and a second blocking projection located between the channel and the resilient hinge to inhibit movement of the plunger rod out of the channel. This may make insertion of the plunger rod into the correct location in the channel easier for a user, and prevent damage to the resilient hinge.

The dose divider may be integrally formed from a single piece of material, optionally wherein the material is a polymer. This may provide a dose divider that is simple and inexpensive to manufacture, and easy to modify.

The dose divider may have been formed by additive manufacturing or a moulding process.

The resilient hinge may have been formed from a polymer.

The resilient hinge may comprise a curved portion coupled at an end of a respective straight portion.

The resilient hinge may comprise a curved portion coupled at either end to a respective straight portion. This may provide a compact hinge with good flexibility and resiliency characteristics.

The curved portion may curve away from a front end of the dose divider such that a vertex of the curved portion is at a rear of the resilient hinge.

The resilient hinge may be arcuate.

The resilient hinge may be a living hinge integrally formed with the first arm and the second arm.

According to a second aspect of the present disclosure, there is provided a medicament delivery device comprising a dose divider according to any previous disclosure and a syringe, wherein the syringe comprises the plunger rod and the dose divider is removably coupled to the plunger rod.

The syringe may comprise an atomizer and be configured for intranasal delivery of a medicament.

The medicament may be an RSV vaccine.

The medicament delivery device may be configured to deliver the RSV vaccine intranasally with about ½ dose delivered to each nostril of a patient.

The medicament delivery device may be configured to deliver the intranasal dose of the RSV vaccine in about 0.2 mL, wherein about 0.1 mL is delivered to each nostril.

The medicament delivery device may be configured to deliver a second dose intranasally with about ½ dose delivered to each nostril of a patient.

The medicament delivery device may be configured to deliver the second dose of the RSV vaccine in about 0.2 mL, wherein about 0.1 mL is delivered to each nostril.

The medicament delivery device may be configured to deliver an average droplet size Dᵥ₅₀ of about 10-120 μm.

An average droplet size Dᵥ₅₀ delivered to each nostril may be about 10-120 μm, about 30-110 μm, about 50-110 μm, about 70-110 μm, or about 80-110 μm.

The medicament delivery device may be configured to deliver an average shot volume to each nostril between about 85 L to about 120 μL, about 90 μL to about 115 μL, or about 95 μL to about 115 μL.

According to a third aspect of the present disclosure, there is provided a method of using a dose divider according to any previous disclosure, the method comprising:
while the dose divider is coupled to a plunger rod of a syringe, moving the plunger rod in a first range of motion with respect to a container of the syringe to expel a first dose of a medicament contained within the syringe;
removing the dose divider from the plunger rod; and
while the dose divider is removed from the plunger rod, moving the plunger rod in a second range of motion with respect to the container to expel a second dose of a medicament contained within the syringe.

According to a fourth aspect of the present disclosure, there is provided a method of using a dose divider according to any previous disclosure, the method comprising: moving the dose divider from the first configuration to the second configuration by applying a force to the first arm and the second arm such that the first arm pivots in a first direction with respect to the second arm using the resilient hinge.

The first arm and the second arm of the dose divider may each comprise a respective rear portion, wherein applying the force to the first arm and the second arm may comprise applying the force to the rear portion of the first arm and the rear portion of the second arm such that the rear portion of the first arm and the rear portion of the second arm are pivoted towards each other and the front portion of the first arm and the front portion of the second arm pivot away from each other.

The rear portion of the first arm may comprise a first protrusion and the rear portion of the second arm may comprise a second protrusion,
the method may further comprise:
moving the rear portion of the first arm towards the rear portion of the second arm such that the first protrusion and the second protrusion are brought into contact with each other to limit further movement of the rear portion of the first arm towards the rear portion of the second arm.

The method may further comprise: subsequent to moving the dose divider from the first configuration to the second configuration, moving the dose divider from the second configuration to the first configuration by releasing the force applied to the first arm and the second arm such that the first arm pivots with respect to the second arm using the resilient hinge in a second direction opposite to the first direction.

The method may further comprise: while the dose divider is in the second configuration, coupling the dose divider to a plunger rod of a syringe or removing the dose divider from the plunger rod of the syringe.

The method may further comprise: while the dose divider is coupled to a plunger rod of a syringe, moving the plunger rod in a first range of motion with respect to a container of the syringe to expel a dose of a medicament contained within the syringe.

According to a fifth aspect of the present disclosure, there is provided a system comprising:
a vial containing a medicament;
a syringe comprising a container and a plunger rod;
a needle configured to be coupled to the syringe such that the medicament can be drawn via the needle from the vial into the container;
an atomizer configured to be coupled to the syringe; and
a dose divider according to any preceding disclosure configured to be coupled to the plunger rod.

The medicament may comprise a vaccine.

The vaccine may be an RSV vaccine.

The syringe may be configured for intranasal delivery of a medicament.

The system may be configured to deliver the RSV vaccine intranasally with about ½ dose delivered to each nostril of a patient.

The system may be configured to deliver the intranasal dose of the RSV vaccine in about 0.2 mL, wherein about 0.1 mL is delivered to each nostril.

The system may be configured to deliver a second dose intranasally with about ½ dose delivered to each nostril of a patient.

The system may be configured to deliver the second dose of the RSV vaccine in about 0.2 mL, wherein about 0.1 mL is delivered to each nostril.

The system may be configured to deliver an average droplet size Dᵥ₅₀ of about 10-120 μm.

An average droplet size Dᵥ₅₀ delivered to each nostril may be about 10-120 μm, about 30-110 μm, about 50-110 μm, about 70-110 μm, or about 80-110 μm.

The system may be configured to deliver an average shot volume to each nostril between about 85 μL to about 120 μL, about 90 μL to about 115 μL, or about 95 L to about 115 μL.

According to a sixth aspect of the present disclosure, there is provided method of using a system, the system comprising:
a vial containing a medicament;
a syringe comprising a container and a plunger rod;
a needle configured to be coupled to the syringe such that the medicament can be drawn via the needle from the vial into the container;
an atomizer configured to be coupled to the syringe; and
a dose divider comprising:
   a first arm and a second arm opposite the first arm, each comprising a respective front portion, wherein the front portion of the first arm and the front portion of the second arm form a channel therebetween for receiving the plunger rod; and
   a resilient hinge pivotably coupling the first arm and the second arm such that the dose divider is reversibly movable between a first configuration and a second configuration,
   wherein, when the dose divider is in the second configuration, the dose divider is configured to receive the plunger rod in the channel through an opening formed between the front portion of the first arm and the front portion of the second arm,
   wherein, when the dose divider is in the first configuration, the channel has a shape configured to receive a specific outer shape of the plunger rod, and
   wherein the front portion of the first arm and the front portion of the second arm are closer when the dose divider is in the first configuration than when the dose divider is in the second configuration,
      wherein the method comprises:
while the needle is coupled to the syringe, drawing medicament into the container of the syringe from the vial via the needle;
coupling the dose divider to the plunger rod;
removing the needle from the syringe;
coupling the atomizer to the syringe;
while the dose divider is coupled to the plunger rod and the atomizer is coupled to the syringe, moving the plunger rod in a first range of motion with respect to the container of the syringe to expel a first dose of the medicament from the container and out of the atomizer;
removing the dose divider from the plunger rod after expelling the first dose of the medicament; and
while the dose divider is removed from the plunger rod and the atomizer is coupled to the syringe, moving the plunger rod in a second range of motion with respect to the container to expel a second dose of the medicament from the container and out of the atomizer.

The atomizer may be at least partially inserted into a first nostril of a patient while the first dose of the medicament is expelled, and wherein the atomizer may be at least partially inserted into a second nostril of the patient while the second dose of the medicament is expelled.

The method may further comprise coupling the needle to the syringe prior to drawing the medicament into the syringe from the vial.

The medicament may comprise a vaccine.

The vaccine may be an RSV vaccine.

The method may comprise delivering the RSV vaccine intranasally with about ½ dose delivered to each nostril of a patient.

The method may comprise delivering the intranasal dose of the RSV vaccine in about 0.2 mL, wherein about 0.1 mL is delivered to each nostril.

The method may comprise delivering a second dose intranasally with about ½ dose delivered to each nostril of a patient.

The method may comprise delivering the second dose of the RSV vaccine in about 0.2 mL, wherein about 0.1 mL is delivered to each nostril.

The method may comprise delivering an average droplet size Dᵥ₅₀ of about 10-120 μm.

An average droplet size Dᵥ₅₀ delivered to each nostril may be about 10-120 μm, about 30-110 μm, about 50-110 μm, about 70-110 μm, or about 80-110 μm.

The method may comprise delivering an average shot volume to each nostril between about 85 μL to about 120 μL, about 90 μL to about 115 μL, or about 95 μL to about 115 μL.

The resilient hinge may be configured to bias the dose divider towards the first configuration.

The first arm and the second arm may each comprise a respective rear portion, wherein the dose divider may be configured to be moved from the first configuration to the second configuration by a user moving the rear portion of the first arm towards the rear portion of the second arm.

An outer surface of the rear portion of the first arm and an outer surface of the rear portion of the second arm may comprise respective grip formations.

Each grip formation may comprise a plurality of ridges.

The dose divider may further comprise a stop formation located on at least one of the rear portion of the first arm and the rear portion of the second arm, wherein the stop formation may be located between the first arm and the second arm to limit movement of the rear portion of the first arm towards the rear portion of the second arm.

The stop formation may comprise a first protrusion located on the rear portion of the first arm and a second protrusion located on the rear portion of the second arm, wherein the first protrusion and the second protrusion may be configured to be brought into contact with each other as the rear portion of the first arm is moved towards the rear portion of the second arm, to limit further movement of the rear portion of the first arm towards the rear portion of the second arm.

The first protrusion may comprise a groove configured to receive at least a portion of the second portion when the first protrusion and the second protrusion are brought into contact with each other.

The front portion of the first arm and the front portion of the second arm may each comprise a respective guide surface for guiding the plunger rod through the opening towards the channel.

The dose divider may further comprise at least one of a first blocking projection and a second blocking projection located between the channel and the resilient hinge to inhibit movement of the plunger rod out of the channel.

The dose divider may be integrally formed from a single piece of material, optionally wherein the material may be a polymer.

The dose divider may have been formed by additive manufacturing or a moulding process.

The resilient hinge may be formed from a polymer.

The resilient hinge may comprise a curved portion coupled at either end to a respective straight portion.

The resilient hinge may be a living hinge integrally formed with the first arm and the second arm.

These and other aspects of the disclosure will be apparent from and elucidated with reference to the embodiments described hereinafter.

### BRIEF DESCRIPTION OF THE FIGURES

Embodiments will now be described, by way of example only, with reference to the accompanying drawings, in which:

FIG. 1 is a perspective view of a syringe suitable for use in embodiments of the present disclosure, with a plunger rod shown in an initial position;

FIG. 2 is a side view of the syringe of FIG. 1;

FIG. 3 is a side view of the syringe of FIGS. 1 and 2 after the plunger rod has been moved from the initial position;

FIG. 4 is a perspective view of a dose divider according to embodiments of the present disclosure, with the dose divider in a first configuration;

FIG. 5 is a top view of the dose divider of FIG. 4, in the first configuration;

FIG. 6 is a top view of the dose divider of FIG. 4, in a second configuration;

FIG. 7 is a perspective view of the dose divider of FIGS. 4 to 6 being attached to the syringe of FIGS. 1 to 3 while in the second configuration;

FIG. 8 is a perspective view of the dose divider of FIG. 7 in the first configuration, attached to the plunger rod of the syringe;

FIG. 9 is a side view of the dose divider and syringe of FIG. 8;

FIG. 10 is a cross-sectional side view of the dose divider and syringe of FIGS. 8 and 9, with the dose divider coupled to the syringe and the plunger rod in an initial position;

FIG. 11 is a cross-sectional side view of the dose divider and syringe of FIG. 10, with the plunger rod now in an intermediate position;

FIG. 12 is a cross-sectional side view of the dose divider and syringe of FIG. 11, with the plunger rod now in an end position; and

FIG. 13 is flow chart representing a method of using a dose divider and syringe.

### DETAILED DESCRIPTION

Aspects of the present disclosure provide an improved dose divider, also known as a dose divider clip. The dose divider may be easy to control by a user, for example when removing or attaching the dose divider to a plunger rod of a syringe. The dose divider may in particular be easier to control for users with reduced dexterity. The dose divider may be easier to reuse; for example the dose divider may allow for repeated attachment and detachment to the plunger rod of a syringe. The dose divider may be able to be coupled to many different types of plunger rod, having different cross-sections. The dose divider may be able to be used flexibly, for example it may be able to be attached at many different positions along a plunger rod. The dose divider may be easier to reposition on a plunger rod, for example from a position near a proximal end of the plunger rod towards a position near a distal end of the plunger rod.

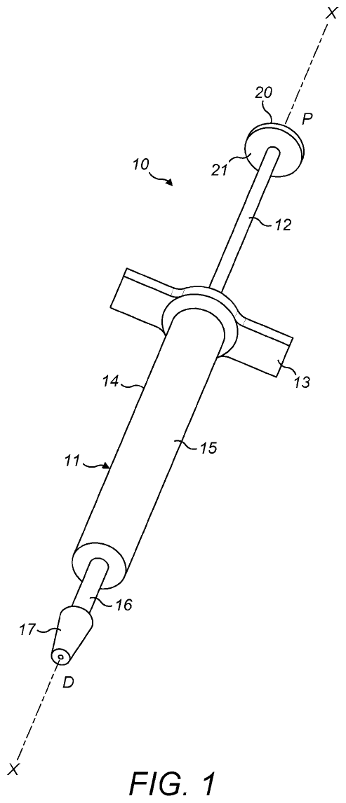

FIGS. 1 to 3 show a syringe 10 suitable for use in various embodiments of the present disclosure. The syringe 10 has a proximal end P and a distal end D, and is generally elongate with a central axis X-X. The syringe 10 comprises a medicament container 11 (hereafter “container 11”), an actuator 12 in the form of a plunger rod or piston rod 12, and optionally a finger grip 13 secured to a proximal end of the container 11. The container 11 is elongate and comprises an outer side wall 14 defining a medicament chamber 15 for containing a medicament. The container 11 is open at a proximal end P and includes an outlet 16 at the distal end D opposite to the proximal end of the container 11. The term “distal” refers to a location that is relatively closer to the outlet 16 and from which medicament is delivered in use, and the term “proximal” refers to a location that is relatively further away from the outlet 16. A distal region of the plunger rod 12, received within the container 11, may be in sealing contact with an inside wall of the container 11, to prevent egress of medicament through the open proximal end P of the container 11. The proximal end of the plunger rod 12 includes an end surface 20 which, in use, a user presses to dispense medicament (described in more detail below). The end surface 20 extends beyond the plunger rod 12 in at least one direction perpendicular to the axis X-X. As such, a distal face 21 of the end surface 20 which opposes the proximal end of the container 11 may eventually contact the proximal end of the container 11 as the plunger rod 12 is pushed into the chamber 15, preventing further movement of the plunger rod 12 into the chamber 15.

The outlet 16 comprises a narrowing of the container 11 into a nozzle. An atomizer 17 may be provided on the outlet 16. The atomizer 17 and/or outlet 16 may comprise a connector such as a Luer lock to sealingly connect the atomizer 17 to the outlet 16. In some examples, the atomizer 17 may be formed to facilitate insertion of the outlet into an orifice into which medicament is to be delivered, for example a nostril. The atomizer 17 may be configured with a medicament passage configured to atomise a liquid medicament expelled through the outlet 16 and through the atomizer 17.

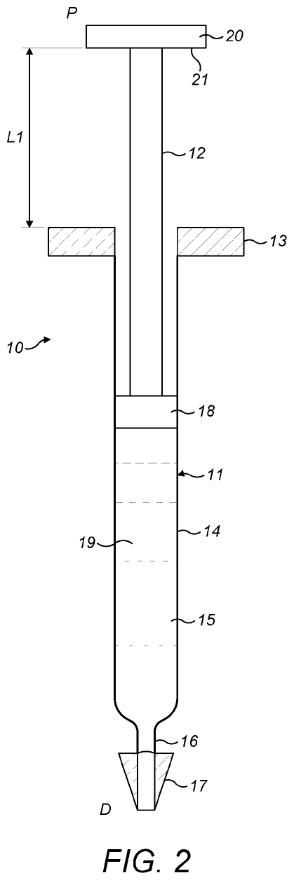

The atomizer 17 may have a conical shape as shown in FIG. 1 and FIG. 2, with the outer surface of the atomizer 17 tapering with a decreasing width from a proximal end of the atomizer 17 (at which the atomizer 17 is coupled to the outlet 16) towards a distal end of the atomizer 17 (at which the atomizer 17 is to be first inserted into the orifice). The conical shape of the atomizer 17 may allow atomized medicament to be more consistently and accurately directed towards a desired target when inserted into an orifice such as a nostril. For example, medicament may be more consistently and accurately directed towards the top of a nasal passage of a patient when the atomizer 17 is inserted through a nasal valve and into a nasal cavity.

The atomizer 17 may be configured to deliver an average droplet size Dᵥ₅₀ of about 10-120 μm. The average droplet size Dᵥ₅₀ delivered to each nostril may be about 10-120 μm, about 30-110 μm, about 50-110 μm, about 70-110 μm, or about 80-110 μm.

The atomizer 17 may have a tip diameter at its distal end of between approximately 4 mm and 5 mm, for example approximately 4.3 mm. The atomizer 17 may have a dead space of less than approximately 0.3 mL, for example less than approximately 0.2 mL. In some examples the dead space may be approximately 0.15 mL.

The atomizer 17 may be formed from a medical-grade polycarbonate material. Other suitable materials may be envisaged.

The atomizer 17 may be ISO-594 compliant (with a suitable Luer lock) and the syringe 10 may also be ISO-594 compliant (with a suitable Luer lock), for coupling the syringe 10 to the atomizer 17.

The container 11 described herein may be any suitable shape or configuration, such as a hollow tube or other body. In some examples, a piston 18 may be provided within the medicament chamber 15 in sealing contact with the inside wall of the container 11. In such an arrangement, the piston 18 may be attached to the distal end of the plunger rod 12 (see FIG. 2), and the plunger rod 12 may therefore not need to make sealing contact with the inside wall of the container 11.

In use, medicament 19 is provided within the medicament chamber 15, and remains in the chamber 15 due to the seal of the plunger rod 12 and/or piston 18 at the proximal end of the chamber 15, and the narrow outlet 16 at the distal end of the chamber 15, which may be provided with a cap, valve, temporary rupturable seal, or other closure. Alternatively, the narrow outlet 16 alone may prevent medicament 19 escaping from the distal end of the chamber 15. The medicament 19 may be expelled in one or more doses from the chamber 15 by linearly moving the plunger rod 12 in a distal direction into the chamber 15, thereby reducing the volume of the chamber 15 between the outlet 16 and the plunger rod 12 and/or piston 18 to force the medicament 19 out of the outlet 16 (and atomizer 17 if present).

FIG. 2 is a cross-sectional side view of the syringe 10 of FIG. 1 in an initial state, showing the plunger rod 12 in an initial position with respect to the container 11. The syringe 10 of FIG. 2 may be in a state prior to medicament delivery, with the chamber 15 containing medicament 19 equivalent to one or more doses to be delivered, and the distal face 21 of the end surface 20 located a distance L1 from the proximal end of the container 11 along the central axis X-X.

In use, a user may apply a force to the plunger rod 12 via the end surface 20 in a direction from the proximal end towards the distal end of the container 11, causing the plunger rod 12 to translate with respect to the container 11. This translation of the plunger rod 12 towards the distal end of the container 11 and further into the chamber 15 causes the plunger rod 12 and/or piston 18 to apply a force to the medicament 19 in the chamber 15, causing at least a portion of the medicament 19 to be expelled from the chamber 15 via the outlet 16 and optional atomizer 17.

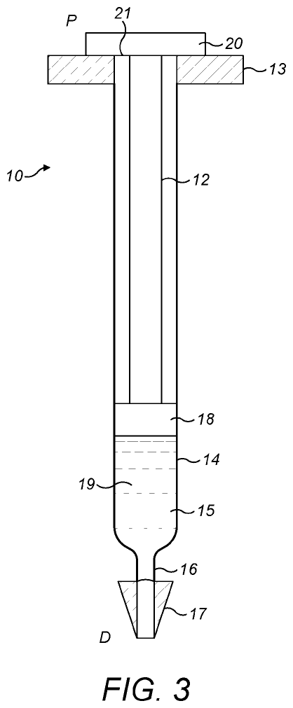

FIG. 3 is a cross-sectional side view of the syringe 10 of FIG. 2 in a final state, after the plunger rod 12 has been moved from its initial position with respect to the container 11 to a final position with respect to the container 11, expelling at least a portion of the medicament 19 from the chamber 15 in the process. FIG. 3 shows a portion of the medicament 19 still remains in the chamber 15, however in other examples substantially all of the medicament 19 may have been ejected from the chamber 15.

FIG. 3 shows the plunger rod 12 and end surface 20 have been moved a distance equal to L1 parallel to the central axis X-X of the syringe 10 such that the distal face 21 of the end surface 20 now abuts the proximal end of the container 11, preventing further translational movement of the plunger rod 12 into the container 11 and chamber 15. However, in other examples further translational movement of the plunger rod 12 into the container 11 and chamber 15 may be prevented once a distal end of the plunger rod 12 or the piston 18 abuts the distal end of the chamber 15, which may occur without the distal face 21 of the end surface 20 abutting the proximal end of the container 11.

The amount of medicament 19 expelled from the syringe 10 during the transition between the initial state shown in FIG. 2 and the final state shown in FIG. 3 may be equivalent to a dose of medicament. However, in other examples the amount of medicament 19 expelled may be equivalent to two doses or a greater number of doses than two. In such a scenario, a user of the syringe 10 may wish to expel fewer than all doses of medicament within the syringe 10 during a single medicament delivery procedure. For example, the user may wish to expel a single dose of medicament 19 out of a plurality of doses of medicament 19 contained in the chamber 15. The user may therefore move the plunger rod 12 in a first range of motion across a certain distance towards the container 11 that results in an amount of medicament 19 equivalent to one dose (or a different required number of doses) being expelled from the syringe 10, before stopping further movement of the plunger rod 12. As an example, this certain distance could be less than L1 shown in FIG. 2. The user may then, at a subsequent moment in time, perhaps after moving the syringe 10 to a different location in a patient, expel one or more subsequent doses of medicament 19 by moving the plunger rod 12 in a second range of motion across another distance towards the distal end of the container 11. As such, the syringe 10 may be used to dispense more than one discrete dose of medicament 19.

As an example, if the amount of medicament 19 expelled from the syringe 10 during the transition between the initial state shown in FIG. 2 and the final state shown in FIG. 3 is equivalent to two doses of medicament 19, the user may move the plunger rod 12 a distance equivalent to half of L1 to expel a first dose and then move the plunger rod 12 another distance equivalent to half of L1 to expel a second dose. If the syringe 10 is being used for intranasal vaccine delivery, the user may expel the first dose via a first nostril of a patient and expel the second dose via a second nostril of the patient.

It may be difficult for a user to accurately divide doses of a medicament 19 by moving the plunger rod 12 of a syringe 10 by predetermined distances that are fractions of the total movable extent of the plunger rod 12. For example, it can be difficult to accurately judge how far to move the plunger rod 12. This could result in accidental underdosing or overdosing, with potential consequences for the patient and/or wastage of medicament.

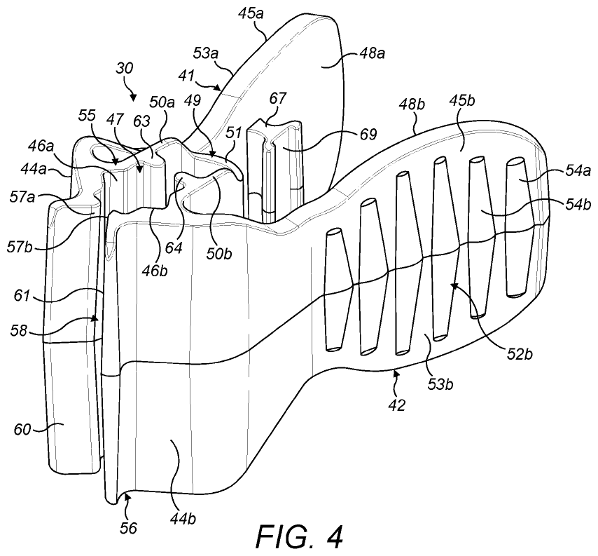

FIGS. 4 to 6 show a dose divider 30 (dose divider element or dose divider clip) according to one or more embodiments of the present disclosure, wherein the dose divider 30 can be used for dividing doses of medicament 19 contained in a syringe 10. The dose divider 30 is configured to be removably coupled to the plunger rod 12 of a syringe 10 and may allow a user to more easily and more accurately expel two or more individual doses of medicament 19 from the syringe 10. The syringe 10 may be the syringe 10 as previously described in relation to FIGS. 1 to 3, however it should be noted that the syringe 10 illustrated in FIGS. 1 to 3 is shown by way of example and that the dose divider 30 may be used with any suitable syringe 10.

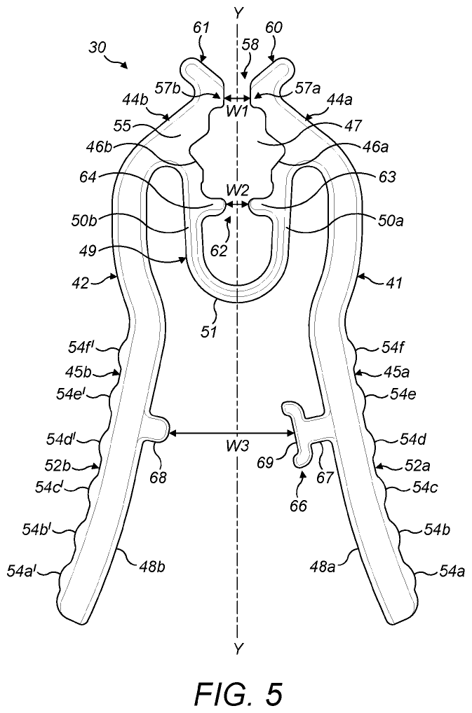

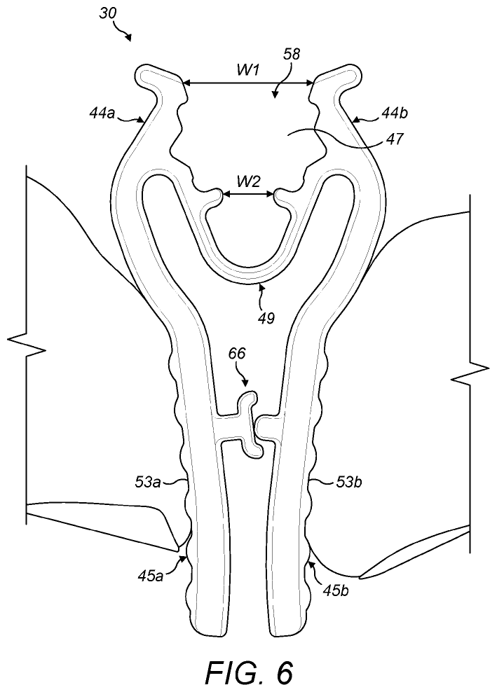

FIG. 4 shows a perspective view of the dose divider 30, while FIGS. 5 and 6 show top views of the dose divider 30 of FIG. 4. FIG. 5 shows the dose divider 30 in a first configuration while FIG. 6 shows the dose divider 30 in a second configuration, as explained later.

As illustrated in FIGS. 4 and 5, the dose divider 30 is generally elongate, having a front end and a rear end with respect to a central axis Y-Y of the dose divider 30, the front end at the top of FIG. 5, near the opening 58, and the rear end at the bottom of FIG. 5, near the rear portions 45a, 45b.

The dose divider 30 comprises a first arm 41 and a second arm 42 arranged either side of the central axis Y-Y. The first arm 41 and the second arm 42 are spaced apart from and located opposite to one another, and each may extend substantially parallel to the central axis Y-Y.

The first arm 41 comprises a front portion 44a located at a front end of the first arm 41 and a rear portion 45a located at a rear end of the first arm 41. Similarly, the second arm 42 comprises a front portion 44b located at a front end of the second arm 42 and a rear portion 45b located at a rear end of the first arm 41. The term “front” refers to a location that is relatively closer to the front end of the dose divider 30, and the term “rear” refers to a location that is relatively further away from the front end.

The front portion 44a of the first arm 41 is arranged opposite the front portion 44b of the second arm 42 such that the front portions 44a, 44b are either side of the axis Y-Y and such that an inner surface 46a of the front portion 44a of the first arm 41 faces an inner surface 46b of the front portion 44b of the second arm 42. The front portion 44a of the first arm 41 and the front portion 44b of the second arm 42 form a channel 47 therebetween, for receiving the plunger rod 12 of a syringe 10, as discussed later. The channel 47 is formed between the inner surfaces 46a, 46b.

The rear portion 45a of the first arm 41 is arranged opposite the rear portion 45b of the second arm 42 such that the rear portions 45a, 45b are either side of the axis Y-Y and such that an inner surface 48a of the rear portion 45a of the first arm 41 faces an inner surface 48b of the rear portion 45b of the second arm 42.

The first arm 41 and the second arm 42 are pivotably coupled by a resilient hinge 49 such that the first arm 41 is able to be pivoted with respect to the second arm 42 via the resilient hinge.

The resilient hinge 49 pivotably couples the first arm 41 and the second arm 42 such that the dose divider 30 is reversibly movable between a first configuration, in which the first arm 41 is in a first position relative to the second arm 42, and a second configuration, in which the first arm 41 is in a second position relative to the second arm 42. The dose divider 30 is movable from the first configuration to the second configuration by a user moving the rear portions 45a, 45b closer together, which movement causes the first arm 41 to pivot with respect to the second arm 42 using the resilient hinge 49, such that the front portions 44a, 44b move further apart. The resilient nature of the resilient hinge 49 biases the dose divider from the second configuration towards the first configuration such that removal of the force bringing the rear portions 45a, 45b together causes the rear portions 45a, 45b to move apart, the first arm 41 to pivot with respect to the second arm 42 in an opposite direction to before, and the front portions 44a, 44b to move closer together. The first and second configurations are discussed in more detail later.

The resilient hinge 49 may be coupled at one end of the resilient hinge 49 to the first arm 41 at a position between the front portion 44a and the rear portion 45a of the first arm 41, and coupled at the other end of the resilient hinge 49 to the second arm 42 at a position between the front portion 44b and the rear portion 45b of the second arm 42.

The resilient hinge 49 may be arcuate, as shown in FIGS. 4 and 5. In particular, the resilient hinge 49 may curve in a plane parallel to the axis Y-Y, with the vertex of the curve arranged towards the rear of the resilient hinge 49. This may allow the resilient hinge 49 to provide good resiliency characteristics while remaining compact, and therefore also allowing for a compact dose divider 30.

FIG. 4 shows the resilient hinge 49 comprising a pair of substantially parallel straight portions 50a, 50b that are opposite to and spaced apart from each other, either side of the axis Y-Y. The straight portions 50a, 50b extend in a direction substantially parallel to the axis Y-Y. A first straight portion 50a of the pair of straight portions 50a, 50b is coupled at a front end thereof to the first arm 41, at a position between the front portion 44a and the rear portion 45a of the first arm 41. A second straight portion 50b of the pair of straight portions 50a, 50b is coupled at a front end thereof to the second arm 42, at a position between the front portion 44b and the rear portion 45b of the second arm 42. FIG. 5 shows the first straight portion 50a and the second straight portion 50b each bending away from the central axis Y-Y nearer their respective front ends, adjacent to where they are coupled to the respective first arm 41 and second arm 42.

The resilient hinge 49 shown in FIG. 5 further comprises a curved portion 51 coupling the rear ends of the straight portions 50a, 50b. The curved portion 51 curves away from the front end of the dose divider 30 such that the vertex of the curved portion is at the rear of the resilient hinge 49.

The resilient hinge 49 may be a living hinge, integrally formed with the first arm 41 and the second arm 42. This results in a simple hinge that is easy to form and can eliminate the need for more complicated elements such as metal springs, bearings and the like. In some examples the resilient hinge 49 may be formed from a polymer such as polylactic acid (PLA). This may provide a resilient hinge 49 that is simple to manufacture while having good flexibility and resiliency characteristics.

The rear portion 45a of the first arm 41 and the rear portion 45b of the second arm 42 may each have a respective grip formation 52a, 52b arranged on their respective outer surfaces 53a, 53b, at a location to be gripped by a user when moving the dose divider 30 between the first and second configurations or attaching or removing the dose divider 30 from a plunger rod 12. The grip formations 52a, 52b provide an improved gripping surface for the user to hold the rear portions 45a, 45b, for example by providing increased friction between the rear portions 45a, 45b and the user's fingers.

FIGS. 4 and 5 show each grip formation 52a, 52b comprising a plurality of projections such as ridges 54a-f, 54a′f′. Each ridge 54a-f, 54a′f′ extends along the outer surface 53a, 53b of its respective rear portion 45a, 45b in a direction substantially perpendicular to the central axis Y-Y, with the ridges 54a-f, 54a′f′ being spaced from each other in a direction substantially parallel to the central axis Y-Y. Such an arrangement can provide improved friction and grip for a user coupling and removing the dose divider 30 from a syringe 10, as discussed later. FIGS. 4 and 5 show each grip formation 52a, 52b comprising six ridges 54a-f, 54a′f′, however in other examples any number of ridges 54a-f, 54a′f′ could be used for either grip formation 52a, 52b. In other examples, one or both grip formations 52a, 52b may comprise different features to ridges 54a-f, 54a′f′, for example, but not limited to, bumps, grooves, adhesive surfaces and/or high friction surfaces such as rubber. In some examples, a grip formation 52a, 52b may be provided on only one of the rear portions 45a, 45b.

As discussed previously, the front portions 44a, 44b of the dose divider 30 form an elongate channel 47 therebetween for receiving the plunger rod 12 when the dose divider 30 is coupled to the plunger rod 12. The channel 47 extends from a top surface 55 of the dose divider 30 to a bottom surface 56 of the dose divider 30 and is open at both ends so that the plunger rod 12 may be received in the channel 47 and the dose divider 30 may be slid along the plunger rod 12, with the plunger rod 12 translating through the channel 47.

The top surface 55 is formed from top surfaces of the front portions 44a, 44b surrounding the one end of the channel 47, and the bottom surface 56 is formed from bottom surfaces of the front portions 44a, 44b surrounding the other end of the channel 47. A distance L3 between the top surface 55 and the bottom surface 56, corresponding to a height of the dose divider 30, may in some examples be between 16 mm and 19 mm, for example a dimension L3 between 17 mm and 18 mm, or more particularly a dimension L3 equal to 17.5 mm.

The height of the dose divider 30 corresponds to a volume of dose to be expelled, as described elsewhere in this application. For example, a dose divider 30 having a dimension L3 equal to 17.5 mm may correspond to a 0.1mL dose of medicament to be expelled. In another example, a dose divider 30 having a dimension L3 equal to 35.0 mm may correspond to a 0.2mL dose of medicament to be expelled, while a dose divider 30 having a dimension L3 equal to 26.25 mm may correspond to a 0.15 mL dose of medicament to be expelled. However other values of dimension L2, dose volume, and ratios of dimension L2 to dose volume may be envisaged. For example, a dose divider 30 having a dimension L3 equal to 20.0 mm may correspond to a 0.2 mL dose of medicament to be expelled or a dose divider 30 having a dimension L3 equal to 10.0 mm may correspond to a 0.2 mL dose of medicament to be expelled.

In some examples, different dose dividers 30 may have different colours corresponding to the dose volume they allow to be expelled (i.e. the colours correspond to the dimension L3 of the dose divider 30). For example, a red-coloured dose divider 30 (or at least part of the dose divider 30 being coloured red) could indicate that the dose divider 30 has a dimension L3 equal to 17.5 mm and therefore corresponds to a 0.1 mL dose of medicament to be expelled, while a green-coloured dose divider 30 (or at least part of the dose divider 30 being coloured green) could indicate that the dose divider 30 has a dimension L3 equal to 35.0 mm and therefore corresponds to a 0.2 mL dose of medicament to be expelled. The colours, dimensions L3 and dose volumes are given as examples and it is envisaged that other colours, L3 dimensions or dose volumes and/or combinations thereof can be used.

In some examples, dose dividers 30 corresponding to different dose volumes may be differentiated in a different way to colour (or in addition to colour), for example by having a marking formed on the dose divider 30 that represents the dose volume, or forming at least part of the dose divider 30 from a particular material and/or with a particular surface characteristic indicative of the dose volume.

The channel 47 may have a substantially constant cross-section along its length. The cross-section of the channel 47 may closely correspond to the cross-section of the plunger rod 12 such that, when the dose divider 30 is coupled to the plunger rod 12, the plunger rod 12 is securely received in the channel 47. However, the cross-section of the channel 47 may be slightly larger than the cross-section of the plunger rod 12 in each dimension to allow the plunger rod 12 to freely slide through the channel 47. FIGS. 4 and 5 show the channel having a star-shaped cross-section formed from a series of ridges and grooves formed along the length of the channel 47, however it can be envisaged that different cross-sections may instead be provided for the channel 47. The channel 47 will have a specific shape configured to receive a specific outer shape of the plunger rod 12, allowing for the plunger rod 12 to be snugly received in the channel 47.

The front portions 44a, 44b of the first and second arms 41, 42 are separated by a gap at their front ends, forming an opening 58. The opening 58 is situated at the front end of the dose divider 30, with the channel 47 to the rear of the opening 58. The opening 58 is configured to receive the plunger rod 12 during coupling of the dose divider 30 to the plunger rod 12. The opening 58 leads to a side of the channel 47 such that the plunger rod 12 may pass through the opening 58 and into the channel 57 as the dose divider 30 is coupled to the plunger rod 12.

Each of the front portions 44a, 44b may comprise a lip 57a, 57b formed on the inner surface 48a, 48b of the respective front portions 44a, 44b, at a front end of said front portions 44a, 44b. Each lip 57a, 57b extends in an elongated manner along a direction from the top surface 55 to the bottom surface 56 of the dose divider 30, parallel to the longitudinal axis of the channel 47. A rear side of each lip 57a, 57b forms part of the wall of the channel 47. The lips 57a, 57b form the opening 58 therebetween.

The opening 58 is elongate, extending parallel to the lips 57a, 57b and to the longitudinal axis of the channel 47. The opening has a width W1 between the lips 57a, 57b, representing the smallest distance between the lips 57a, 57b. The dose divider 30 is configured such that, when the dose divider 30 is coupled to the plunger rod 12 as described later, the lips 57a, 57b inhibit removal of the plunger rod 12 from the channel 47 in a direction towards the front of the dose divider 30 (i.e. perpendicular to axis X-X and parallel to axis Y-Y). This is achieved by forming the dose divider 30 such that, when the dose divider 30 is coupled to the plunger rod 12, the width W1 of the opening 58 is smaller than a corresponding width of the plunger rod 12. As described later in relation to FIG. 6, the width W1 of the opening 58 changes with movement of the dose divider 30 from the first configuration shown in FIG. 5 to the second configuration shown in FIG. 6, due to pivoting of the first arm 41 with respect to the second arm 42 about the resilient hinge 49.

The dose divider 30 may also comprise first and second guide surfaces 60, 61. The first and second guide surfaces 60, 61 are configured to guide the plunger rod 12 through the opening 58 and into the channel 47 during attachment of the dose divider 30 to the plunger rod 12.

FIGS. 4 and 5 show the first guide surface 60 formed at a front end of the front portion 44a of the first arm 41 and the second guide surface 61 formed at a front end of the front portion 44b of the second arm 42. The first guide surface 60 and the second guide surface 61 are inclined with respect to each other in a manner that guides a plunger rod 12 towards the opening 58 as the dose divider 30 is being coupled to the plunger rod 12. The first guide surface 60 and the second guide surface 61 are each angled such that they become closer to each other as one moves along the axis Y-Y in a direction from the front end to towards the rear end of the dose divider 30. As described later, the guide surfaces can be used to assist in guiding the plunger rod 12 into the opening 58 in a manner that may not require the user to apply substantial pressure or use much precision.

The dose divider 30 may comprise a blocking feature 62 arranged to inhibit movement of the plunger rod 12 towards the rear of the dose divider 30 once the plunger rod 12 is located in the channel 47. FIGS. 4 and 5 show the blocking feature 62 comprising a first blocking projection 63 and a second blocking projection 64 both located to the rear of the channel 47, between the channel 47 and the resilient hinge 49. The first blocking projection 63 is located on the resilient hinge 49, on the first straight portion 50a of the resilient hinge 49, and projects towards the axis Y-Y in a direction substantially perpendicular to the axis Y-Y. The second blocking projection 64 is also located on the resilient hinge 49, this time on the second straight portion 50b of the resilient hinge 49, and also projects towards the axis Y-Y in a direction substantially perpendicular to the axis Y-Y, but in a direction opposite to the first blocking projection 63. The first blocking projection 63 and the second blocking projection 64 are substantially aligned with each other in a plane parallel to the axis Y-Y and are separated by a distance W2. FIGS. 4 and 5 show the first blocking projection 63 and the second blocking projection 64 each taking the form of a respective ridge that extends perpendicular to the channel 47 along its longitudinal axis.

The first blocking projection 63 and the second blocking projection 64 may be dimensioned such that the distance W2 is sufficiently small when the dose divider 30 is in the first configuration to inhibit movement of the plunger rod 12 out of the channel 47 and towards the rear of the dose divider 30. As discussed elsewhere, moving the dose divider 30 from its first configuration to its second configuration will cause the first blocking projection 63 and the second blocking projection 64 to move apart, increasing the distance W2. As such, the first blocking projection 63 and the second blocking projection 64 may also be dimensioned such that the distance W2 remains sufficiently small when the dose divider 30 is in the second configuration to inhibit movement of the plunger rod 12 out of the channel 47 and towards the rear of the dose divider 30.

The dose divider 30 may further comprise a stop formation 66 for limiting movement of the rear portion 45a of the first arm 41 and the rear portion 45b of the second arm 42 towards each other. FIGS. 4 and 5 show the stop formation 66 located at the rear portions 45a, 45b of the first arm 41 and the second arm 42, on the inner surfaces 48a, 48b of the rear portions 45a, 45b. In particular, the stop formation 66 is shown to comprise a first protrusion 67 extending from the inner surface 48a of the rear portion 45a of the first arm 41 and a second protrusion 68 extending from the inner surface 48b of the rear portion 45b of the second arm 42. The first protrusion 67 and the second protrusion 68 are each located approximately midway along the length of their respective rear portions 45a, 45b, however other locations along the length of the respective rear portions 45a, 45b may be used instead.

The first protrusion 67 and the second protrusion 68 each extend towards the central axis Y-Y in substantially opposite directions and are separated by a shortest distance W3. The first protrusion 67 and the second protrusion 68 are arranged such that, as the dose divider 30 is moved from the first configuration towards the second configuration by moving the rear portions 45a, 45b of the first arm 41 and second arm 42 together, the first protrusion 67 and the second protrusion 68 move towards each other such that the distance W3 between them decreases, until a point that the first protrusion 67 abuts the second protrusion 68 to prevent further movement of the rear portions 45a, 45b together (i.e. the point at which W3=0). This is illustrated by the second configuration of the dose divider 30 shown in FIG. 6.

By adjusting the dimensions of the stop formation 66, such as the distance the first protrusion 67 and/or second protrusion 68 extends from its respective rear portion 45a, 45b, the amount by which the rear portions 45a, 45b can be brought together is in turn adjusted, along with the amount by which the front portions 44a, 44b can move apart. Providing the dose divider 30 with a stop formation 66 can therefore provide a simple means of adjusting the amount by which the front portions 44a, 44b can move apart, and therefore the amount by which the width W1 of the opening 58 can change, which means the dose divider 30 may be easily tailored to accept plunger rods 12 of different dimensions. The provision of the stop formation 66 to limit relative movement between the rear portions 45a, 45b can also prevent excessive movement of the rear portions 45a, 45b together, which could break or otherwise damage the first and/or second arms 41, 42, and/or the resilient hinge 49.

FIGS. 4 and 5 show the first protrusion 67 and the second protrusion 68 both extending as ridges along the inner surfaces 48a, 48b of the rear portions 45a, 45b, from near the top of the dose divider 30 to near the bottom of the dose divider 30 in a direction perpendicular to the axis Y-Y. However, it can be envisaged that the first protrusion 67 and/or second protrusion 68 take different forms.

In some examples, the stop formation 66 may be located on only one of the first arm 41 and second arm 42. For example, the rear portion 45a of the first arm 41 may comprise the first protrusion 67 extending from the inner surface 48a of the rear portion 45a of the first arm 41, without a corresponding second protrusion 68 present on the second arm 42. In this case, the first protrusion 67 would be configured to make contact with the inner surface 48b of the rear portion 45b of the second arm 42 as the rear portions 45a, 45b are brought together, to prevent further movement of the rear portions 45a, 45b towards each other.

FIGS. 4 and 5 show the first protrusion 67 having a groove 69 configured to receive at least a portion of the second protrusion 68. The groove 69 is formed in the surface of the first protrusion 67 that abuts the second protrusion 68 as the rear portions 45a, 45b are brought together. The groove 69 extends the length of the first protrusion 67 from the top of the first protrusion 67 to the bottom of the first protrusion 67. Providing a groove 69 in the first protrusion 67 can ensure the second protrusion 68 is guided into, and securely received in, the groove 69 as the first protrusion 67 and the second protrusion 68 are brought into contact, reducing the likelihood of misalignment of the first protrusion 67 and the second protrusion 68 which could result in the first protrusion 67 and the second protrusion 68 not making contact.

The dose divider 30 may be formed from a single piece of material. In some examples the dose divider 30 may be formed all or in part from a polymer such as polylactic acid (PLA). PLA is biodegradable and compostable and so forming the dose divider 30 from PLA may reduce its environmental impact in municipal waste streams. Other suitable polymers or materials may be used instead.

A dose divider 30 made from a single material such as a single polymer could be formed using a moulding process such as an injection moulding process using single-or multi-cavity moulds. In some examples, the dose divider 30 may be formed from more than one material. For example, one or more of the grip formations 52a, 52b may be formed of a different material to the remainder of the dose divider 30. For example, one or more of the grip formations 52a, 52b may be formed from a plastic that is softer and/or more pliable than a plastic used to form the remainder of the dose divider 30, which may maintain overall structural integrity of the dose divider 30 while at the same time improving user comfort when the user grips the dose divider 30. A dose divider 30 made from more than one material such as more than one polymer could be formed using a moulding process such as an injection moulding process. Such a dose divider 30 may be formed using overmoulding or insertion moulding.

In some examples, the dose divider 30 may be formed all or in part by an additive manufacturing process, alternatively known as three-dimensional (3D) printing.

FIGS. 4 and 5 show the dose divider 30 in a first configuration, prior to a process of attaching the dose divider 30 to a plunger rod 12. The dose divider 30 is movable between the first configuration and a second configuration, wherein the second configuration of the dose divider is shown in FIG. 6.

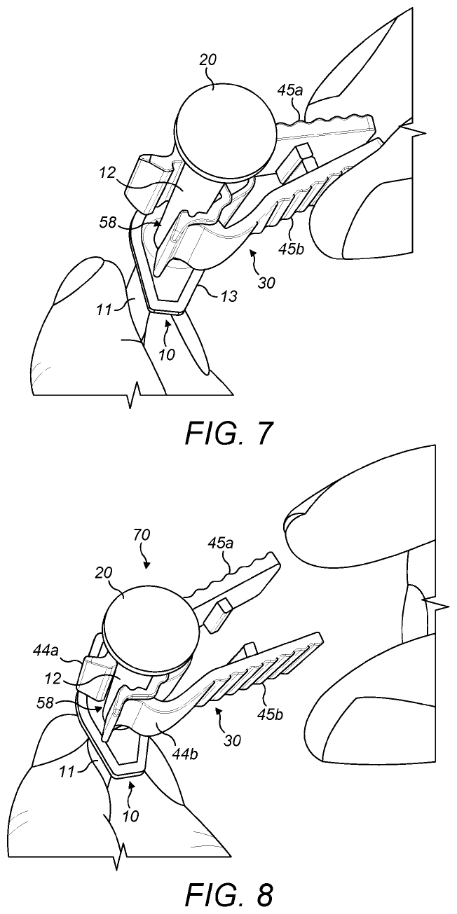

In order to couple the dose divider 30 to the plunger rod 12, a user moves the dose divider 30 from the first configuration (such as shown in FIG. 5) to the second configuration (such as shown in FIG. 6) by applying a compressive force to the outer surfaces 53a, 53b of the rear portions 45a, 45b of the first arm 41 and second arm 42, to bring the rear portions 45a, 45b towards each other, as illustrated in FIGS. 6 and 7.

As shown in FIGS. 6 and 7, a user bringing the rear portions 45a, 45b together by squeezing them between a finger and thumb causes the first arm 41 to pivot with respect to the second arm 42 due to the resilient hinge 49. As the rear portions 45a, 45b are pivoted towards each other, the front portions 44a, 44b move apart. As a result, the lips 57a, 57b move apart, increasing the width W1 of the opening 58. The first and second guide surfaces 60, 61 also move apart, as do the inner surfaces 46a, 46b of the front portions 44a, 44b that form the channel 47. The shape (e.g. cross-section) of the channel 47 will change (e.g. increase in width) as the a user moves the dose divider 30 from the first configuration (such as shown in FIG. 5) to the second configuration (such as shown in FIG. 6). The first blocking projection 63 and the second blocking projection 64 also move apart, increasing the distance W2. Movement of the rear portions 45a, 45b together will eventually be limited by the stop formation 66, wherein the first protrusion 67 and the second protrusion 68 are brought into contact to prevent further relative movement, as shown in FIG. 6.

When the dose divider 30 is in the second configuration, the dose divider 30 is configured to receive the plunger rod 12 in the channel 47 through the opening 48 formed between the front portion 44a of the first arm 41 and the front portion 44b of the second arm 42. That is, a user is able to move the plunger rod 12 into the channel 47 via the opening 48. When the dose divider 30 is in this second configuration, the channel 47 has a shape (i.e. cross-section) that is configured to receive the specific outer shape (i.e. cross-section) of the plunger rod 12. The shape of the channel 47 will be larger than the shape of the plunger rod 12, to allow the plunger rod 12 to be inserted into the channel 47.

The user pivots the first arm 41 with respect to the second arm 42 until the dose divider 30 is in the second configuration, wherein the opening 48 is wide enough to accept the plunger rod 12 (i.e. the width W1 sufficiently corresponds to the width of the plunger rod 12). At this point, the user may bring the plunger rod 12 towards the front end of the dose divider 30 with the longitudinal axis X-X of the plunger rod 12 aligned substantially parallel to the longitudinal axis of the channel 47 and to the opening 58. The user moves the plunger rod 12 through the opening 58 and into the channel 47 such that a portion of the length of the plunger rod 12 is now located in the channel, with the plunger rod 12 co-axial with the channel 47, as shown in FIG. 7.

Before entering the opening 58, the plunger rod 12 may contact one or both of the first and second guide surfaces 60, 61. As the user moves the plunger rod 12 towards the opening 58 of the dose divider 30, reaction forces between the one or both guide surfaces 60, 61 and the plunger rod 12 will guide the plunger rod 12 into alignment with, and eventually through, the opening 58. The guide surfaces 60, 61 therefore allow for greater tolerance in coupling the dose divider 30 to the plunger rod 12, such that the user is not required to be as precise in aligning the plunger rod 12 and dose divider 30.

When the dose divider 30 is in the second configuration, the width W1 of the opening may be wider than a width of the plunger rod 12 to allow the plunger rod 12 to be freely inserted through the opening 58. However, in other examples the width W1 of the opening 58 may be slightly less than the width of the plunger rod 12. In such examples, the user brings the plunger rod 12 to the opening 58 as before and applies force to the plunger rod 12 towards the opening 58.

This force transferred to the guide surfaces 60, 61 or lips 57a, 57b causes the front portions 44a, 44b of the first and second arms 41, 42 to move apart due to flexibility in the first and/or second arms 41, 42, as a result causing the width W1 of the opening 58 to increase.

Continuous application of the force by the user will eventually widen the opening 58 until the plunger rod 12 is able to pass through and into the channel 47. The width W1 of the opening 58 when the dose divider 30 is in the second configuration can be selected by adjusting the dimensions of the stop formation 66, as discussed previously.

As shown in FIG. 6, movement of the dose divider 30 from the first configuration to the second configuration causes the first blocking projection 63 and the second blocking projection 64 to move apart, widening the distance W2 between them. However, the first blocking projection 63 and the second blocking projection 64 may be dimensioned such that, whether the dose divider 30 is in the first configuration or the second configuration, the distance W2 between the first blocking projection 63 and the second blocking projection 64 remains sufficiently small to inhibit movement of the plunger rod 12 out of the channel 47 and towards the rear of the dose divider 30, as illustrated in FIGS. 7 and 8.

Once the plunger rod 12 has been received within the channel 47 of the dose divider 30, the user may release the first arm 41 and second arm 42, at which point the resiliency of the resilient hinge 49 will move the dose divider 30 from the second configuration back towards the first configuration by moving the front portions 44a, 44b together and the rear portions 45a, 45b apart, as illustrated in FIG. 8. The shape (e.g. cross-section) of the channel 47 will change (e.g. decrease in width) as the a user releases the dose divider 30 from the second configuration towards the first configuration, such that the plunger rod 12 is received snugly in the channel 47. When the dose divider 30 is in the first configuration, the channel 47 has a shape (i.e. cross-section) configured to receive a specific outer shape (i.e. cross-section) of the plunger rod 12. That is, the shape of the channel 47 closely corresponds to the shape of the plunger rod 12 such that the plunger rod 12 is received securely in the channel 47.

The syringe 10 and dose divider 30 shown in FIG. 8 may together be referred to as a medicament delivery device 70. The medicament delivery device 70 may be an intranasal atomization delivery device.

FIG. 7 is a perspective view of the dose divider 30 of FIGS. 4 to 6 and the proximal end of a syringe 10 such as a syringe 10 previously described in relation to FIGS. 1 to 3. FIG. 7 shows the dose divider 30 in the second configuration, being coupled to the plunger rod 12 of the syringe 10 in a manner as described previously. The plunger rod 12 has been inserted through the opening 58 of the dose divider 30 such that it is now received within the channel 47, with the plunger rod 12 substantially co-axial with the channel 47. The cross-section of the channel 47 while the dose divider 30 is in the second configuration is wider than the cross-section of the channel 47 while the dose divider 30 is in the first configuration, due to the front portions 44a, 44b of the first arm 41 and second arm 42 being moved apart.

To complete coupling of the dose divider 30 to the plunger rod 12, the user releases the rear portions 45a, 45b of the first arm 41 and the second arm 42, as shown in FIG. 8. The resiliency of the resilient hinge 49 coupling the first arm 41 and the second arm 42 causes the dose divider 30 to move back towards its first configuration, with the front portions 44a, 44b of the first arm 41 and second arm 42 moving back towards each other. This reduces the cross-section of the channel 47, allowing the channel 47 to surround the plunger rod 12 more securely than before. The lips 57a, 57b have moved closer together, reducing the width W1 of the opening 58 and inhibiting movement of the plunger rod 12 back out of the dose divider 30. The medicament delivery device 70 is now ready to deliver a first dose of medicament 19.

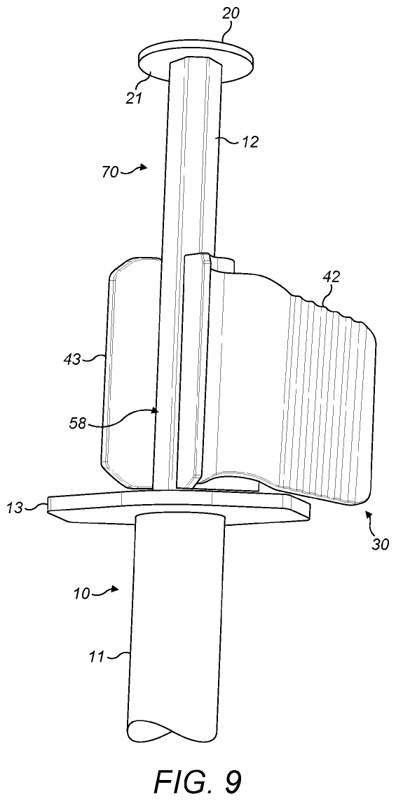

FIG. 9 shows the dose divider 30 and syringe 10 of FIG. 8 from a side perspective, with the dose divider 30 coupled to the plunger rod 12 of the syringe 10. FIG. 9 shows the dose divider 30 coupled at a distal end of the portion of the plunger rod 12 that extends outside the container 11 such that the dose divider 30 abuts the proximal end of the container 11 and the distal face 21 of the end surface 20 is separated from the dose divider 30. However, in other examples the dose divider 30 may be coupled along a different portion of the plunger 12 rod that extends outside the container 11, for example nearer the end surface 20 and not abutting the proximal end of the container 11.

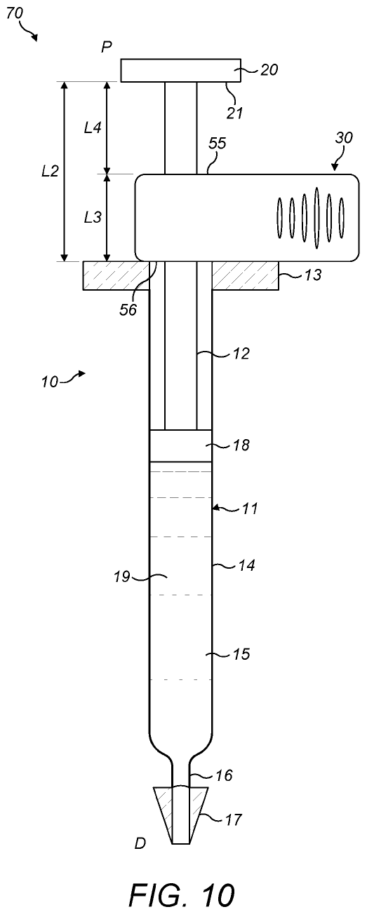

Operation of the dose divider 30 will now be described with reference to FIGS. 10 to 12. The operation will be described in the context of use with an intranasal medicament delivery syringe 10, that is, a syringe 10 for delivery of medicament into the nostril(s) of a patient. However, the concept is not intended to be limited to use with a syringe 10 for intranasal medicament delivery and may equally be applicable to various other forms of syringe 10, in which the medicament is intended to be delivered in two or more separate doses.

In the context of a syringe 10 for intranasal medicament delivery, the volume of medicament such as a vaccine in the syringe 10 is often required to be delivered into each nostril of a patient, often with the volume of medicament divided equally into a dose for each nostril. In use of such a syringe 10, the syringe 10 starts in the initial position shown in FIG. 10. The distal face 21 of the end surface 20 of the plunger rod 12 is located a distance L2 from the proximal end of the container 11 parallel to the central axis X-X. The dose divider 30 has a distance L3 between its top surface 55 and bottom surface 56. A length L4 corresponds to L2 minus L3.

This length L4 is the length of the plunger rod 12 between the distal face 21 of the end surface 20 and the proximal end of the container 11 that is not between the top surface 55 and bottom surface 56 of the dose divider 30. L4 corresponds to the maximum distance that the plunger rod 12 can be moved towards the container 11 while the dose divider 30 is coupled to the plunger rod 12, and therefore corresponds to a maximum amount of medicament 19 that can be expelled during such movement. This amount of medicament 19 may be equivalent to a first dose of the medicament 19.

FIG. 10 shows distance L4 being a continuous length between the distal face 21 of the end surface 20 of the plunger rod 12 and the proximal face of the dose divider 30, however in examples where the dose divider 30 does not initially abut the proximal end of the container 11, L4 may comprise both a length between the distal face 21 of the end surface 20 and the top surface 55 of the dose divider 30 and a length between the bottom surface 56 of the dose divider 30 and the proximal end of the container 11.

In FIG. 10, the plunger rod 12 is in its initial start position and none of the medicament 19 has been expelled from within the chamber 15. Also in this initial position, the dose divider 30 has been coupled to the plunger arm 12 as described previously. The dose divider 30 may be in its first configuration so that it cannot be removed from the plunger 12 without moving it into its second configuration as described previously, or else such removal may be inhibited. This may impede accidental removal of the dose divider 30 from the plunger rod 12. The plunger rod 12 is able to translate relative to the dose divider 30 parallel to the central axis X-X, such that the plunger rod 12 may slide through the channel 47 of the dose divider 30.

The user (who may be a patient themself if the medicament is to be self-administered, or a healthcare professional or other user if the medicament is being administered to the patient by a third party) inserts the atomizer 17 (or outlet 16 if no atomizer 17 is present) into the first nostril of the patient. The user then presses the end surface 20 with their thumb, with their index and middle fingers on the finger grip 13, to move the plunger rod 12 axially in a distal direction towards the distal end of the container 11. This pushes the piston 18 distally within the chamber 15 and expels the medicament 19 out of the outlet 16 and through the medicament passage of the atomizer 17 to atomise the medicament for inhalation by the patient.

The user continues to move the plunger rod 12 distally over this first range of motion until further movement of the plunger rod 12 is inhibited by the dose divider 30. That is, the user continues to move the plunger rod 12 distally over this first range of motion until the distal face 21 of the end surface 20 abuts the dose divider 30 (for example the top surface 55 of the dose divider 30) and dose divider 30 (for example the bottom surface 56 of the dose divider 30) abuts the proximal end of the container 11, at which point the plunger rod 12 cannot move any further in the distal direction. This first range of motion will have a distance equivalent to L4.

If the dose divider 30 was initially coupled to the plunger rod 12 such that the dose divider 30 abutted the proximal end of the container 11, contact between the dose divider 30 and the proximal end of the container 11 would have held the dose divider 30 in position relative to the container 11 while the plunger rod 12 translated in the distal direction through the channel 47 of the dose divider 30 and into the container 11. On the other hand, if the dose divider 30 was initially coupled further towards a proximal end of the plunger rod 12 such that the dose divider 30 did not initially abut the proximal end of the container 11, the dose divider 30 may be moved with the plunger rod 12 as the plunger rod 12 is moved in the distal direction until the dose divider 30 abuts the proximal end of the container 11, after which point the dose divider 30 is fixed relative to the container 11 and further movement of the plunger rod 12 in the distal direction causes the plunger rod 12 to translate through the channel of the dose divider 30 until the until the distal face 21 of the end surface 20 abuts the dose divider 30.

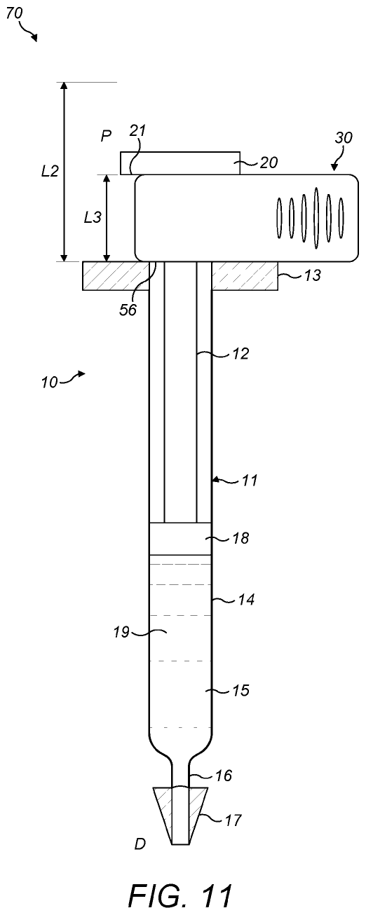

FIG. 11 shows the syringe 10 of FIG. 10 after the plunger rod 12 has been moved distally over the first range of motion. At this point, the plunger rod 12 is in an intermediate position and a first dose of the medicament 19 has been delivered to the patient. At this point, the plunger rod 12 has moved into the container 11 a distance equal to L4 and now the distance L2 between the distal face 21 and the proximal end of the container 11 is approximately equal to L3.

The user then removes the syringe 10 from the first nostril and removes the dose divider 30 from the plunger rod 12. This may be achieved by moving the dose divider 30 to its second configuration by moving the rear portions of the first arm 41 and second arm 42 together as discussed previously, and removing the plunger rod 12 from the channel by sliding it through the opening 58, along the axis Y-Y and away from the rear end of the dose divider 30. The plunger rod 12 is in the intermediate position having been moved over a first range of motion equivalent to L4, but there remains some medicament 19 within the chamber 15 for subsequent dose(s) to be delivered.

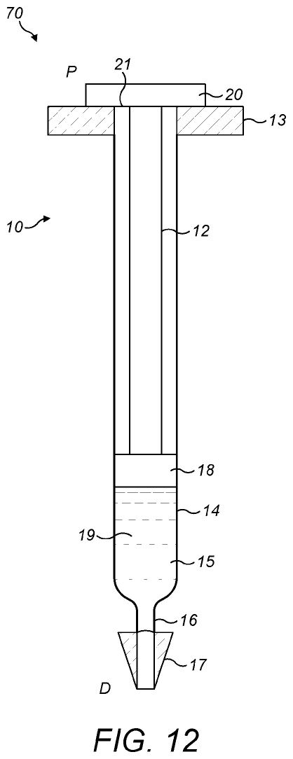

The user now inserts the atomizer 17 into the other nostril of the patient. Since the dose divider 30 has been removed, the plunger rod 12 is no longer prevented from moving further axially in the distal direction by the dose divider 30. The user then presses the end surface 20 again to move the plunger rod 12 axially in a distal direction which pushes the piston 18 distally within the chamber 15 and expels the remaining medicament 19 out of the outlet 16 and through the medicament passage of the atomizer 17 to atomise the medicament for inhalation by the patient. The user continues to move the plunger rod 12 distally over this second range of motion until either the distal face 21 of the end surface 20 abuts the proximal end of the container 11, or the piston 18 abuts the distal end of the chamber 15 and therefore cannot move any further in the distal direction. At this point, the plunger rod 12 is in an end position and the remaining medicament 19 has been expelled as a second and final dose delivered to the patient. FIG. 12 illustrates the syringe 10 of FIG. 11 now in having the dose divider 30 removed and the plunger rod 12 in the end position.

In view of the above, volumes of first and second doses of medicament to be delivered can be controlled by controlling the first and second ranges of motion of the plunger rod 12, which may be controlled by selecting dimension L3 of the dose divider 30 and/or dimension L4 of the syringe 10 as necessary. In embodiments where the first and second doses are to be equal, it will be appreciated that dimension L3 can be selected to be equal to L4.

The dose divider 30 described above provides a simple and effective way to ensure a user is able to divide the medicament delivery process into two (or more) separate doses. The dose divider 30 provides an effective way to ensure the medicament 19 is divided as intended and the user does not accidentally deliver too much/all of the medicament in the first dose.

The dose divider 30 may allow for accurate dose administration without the requirement of the user having fine motor skills and/or visual acuity to confirm dose ejection volume.

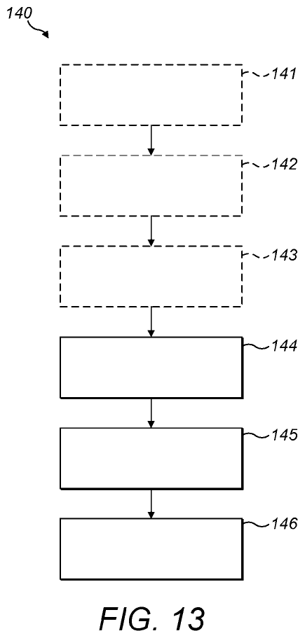

FIG. 13 is a flow chart illustrating a method 140 of using a dose divider 30 with a syringe 10 according to aspects of the present disclosure.

In optional step 141, the user fills the chamber 15 of the syringe 10 with medicament 19 using any suitable technique. For example, the user may couple a needle to the syringe 10 (such as to the outlet 16 of the syringe 10, using any suitable connector) and draw the medicament 19 from a vial and into the chamber 15 within the container 11, via the needle. In other examples, the syringe 10 may pre-filled with medicament 19 before the user receives the syringe 10, e.g. pre-filled by a third party manufacturer.

In optional step 142, the user moves the dose divider 30 from its first configuration to its second configuration, as described previously.

In optional step 143, the user couples the dose divider 30 to the plunger rod 12 of the syringe 10, in a manner as described previously.

In some examples, the dose divider 30 may already be coupled to the syringe 10 before the user receives the syringe 10, e.g. coupled by a third party manufacturer, in which case steps 142 and 143 do not need to be performed.

In step 144, the user expels a first dose of medicament by moving the plunger rod 12 in a first range of motion, in a manner as described previously.

If a needle was coupled to the syringe 10 and used to draw medicament 19 from a vial, the user will have removed the needle from the syringe 10 after drawing the medicament 19 but before expelling the first dose.

If an atomizer 17 is being used to expel the dose, the user will couple the atomizer 17 to the outlet 16 of the syringe 10 if they are not already coupled. This will be done prior to expelling the first dose of medicament 19 and after any needle has been removed from the syringe 10, if a needle was used to draw the medicament 19 as described previously.

In step 145, the user removes the dose divider 30 from the plunger rod 12 of the syringe 10, in a manner as described previously.

In step 146, the user expels a second dose of medicament by moving the plunger rod 12 in a second range of motion, in a manner as described previously.

While certain examples have been described in which the dose divider 30 is able to slide along the plunger rod 12 when coupled to the plunger rod 12, it shall be appreciated that in other examples the dose divider 30 may be configured such that it cannot slide along the plunger rod 12 when coupled to the plunger rod 12, or such movement is substantially inhibited. In such a scenario, a user would couple the dose divider 30 at an initial location along the plunger rod 12 such that the dose divider 30 does not initially abut the proximal end of the container 11 (i.e. there is a separation between the bottom surface 56 of the dose divider 30 and the proximal end of the container 11). The user would therefore be able to move the plunger rod 12 in a distal direction through a first range of motion equivalent to the distance between the dose divider 30 and the proximal end of the container 11, which may be set to correspond to a first dose. The user may then remove the dose divider 30 and move the plunger rod 12 in a distal direction through a second range of motion equivalent to dimension L3 of the removed dose divider 30 (plus any distance between the top surface 55 of the dose divider 30 and the distal face 21 of the end surface 20, if the dose divider was not initially coupled to the plunger rod such that the top surface 55 of the dose divider 30 abutted the distal face 21 of the end surface 20).

It shall be appreciated that aspects of the present disclosure may be used to deliver more than two accurate doses of medicament 19 from a syringe 10. For example, more than one dose divider 30 may be coupled to the plunger rod 12, with each dose divider 30 coupled to the plunger rod 12 in a similar manner as discussed in relation to FIGS. 7 to 9, but with each dose divider 30 spaced along the axial length of the plunger rod 12 (along axis X-X). The dimension L2 of each dose divider 30 may be select to correspond to the amount of medicament 19 that will be expelled when the dose divider 30 is removed from the plunger rod 12 and the plunger rod 12 is moved in the distal direction for the distance L2.

If the plurality of dose dividers 40 are each free to axially slide along the plunger rod 12 then the dose dividers 40 can be removed in any order chosen by the user to expel the dose of medicament corresponding to the dimension L2 of the removed dose divider. This allows for flexibility in the order of doses delivered. On the other hand, if the dose dividers 40 are configured such that they are not able to axially slide along the plunger rod 12 (or such sliding is inhibited), this may force a user must remove the plurality of dose dividers 40 in a particular order if all doses are to be successfully dispensed. More specifically, the dose dividers 40 should be removed starting with the dose divider 30 nearest the proximal end of the container 11, followed by the next dose divider 30 nearest the proximal end of the container 11 and so on, until the dose divider 30 furthest the container 11 is removed, with medicament 19 being dispensed after each removal of a dose divider 40. This scenario can ensure particular doses are administered in a specific order, which may improve safety and/or compliance.

The dose divider 30 can be used with conventional syringes without necessarily adapting the syringes for use with the dose divider 30. Instead, the dose divider 30 may be formed such that it is tailored for use with a particular syringe 10 and/or medicament 19, by adjusting one or more dimensions of the dose divider such as the dimension L3, W1, W2 or W3.

The dose divider 30 as described herein may be easy to use and control by a user, for example when attaching and/or reattaching the dose divider 30 to a plunger rod 12. For example, the provision of a first arm 41 comprising a front portion 44a and a rear portion 45a, a second arm 42 comprising a front portion 44b and a rear portion 45b, and a resilient hinge 49 connecting the first arm 41 and second arm 42 may allow for a dose divider 30 that can be attached or detached from a plunger rod 12 simply by squeezing the rear portions 45a, 45b together in order to cause the from portions 44a, 44b to move apart. As such, the dose divider 30 may be simple to operate. By providing rear portions 45a, 45b that extend a substantial distance away from the front portions 44a, 44b and resilient hinge 49, only a relatively small compression force must be applied to the rear portions 45a, 45b to cause the from portions 44a, 44b to move apart, due to the lever effect provided by the first and second arms 41, 42 and resilient hinge 49. The dose divider 30 may be easier to use and/or control for users with impaired dexterity.

The dose divider 30 as described herein may be easier to reuse. For example, the provision of a first arm 41 comprising a front portion 44a and a rear portion 45a, a second arm 42 comprising a front portion 44b and a rear portion 45b, and a resilient hinge 49 connecting the first arm 41 and second arm 42 may allow for repeated attachment and detachment of the dose divider 30 to the plunger rod 12 of a syringe 10. The dose divider 30 may therefore be reusable rather than single-use. The arrangement of the resilient hinge 49 as described herein may allow for the first arm 41 and second arm 42 to be repeatedly pivoted back and forth with respect to each other without significant degradation of the resilient hinge 49, thereby extending the lifetime of the dose divider 30.

The dose divider 30 as described herein may be able to be used flexibly. For example, it may be able to be attached at many different positions along a plunger rod 12, such as at a proximal end of the plunger rod 12, a distal end of the plunger rod 12, or a mid-point of the plunger rod 12 between the proximal end and the distal end. Furthermore, the dose divider 30 may be easier to reposition on a plunger rod 12 once it has been attached. For example, it may be easier to move the dose divider 30 from a position near the proximal end of the plunger rod 12 to a position nearer the distal end of the plunger rod 12, such as by squeezing the rear portions 45a, 45b together, sliding the dose divider 30 along the plunger rod 12, then releasing the rear portions 45a, 45b. Such flexibility may be brought about by the provision of the first arm 41 comprising a front portion 44a and a rear portion 45a, the second arm 42 comprising a front portion 44b and a rear portion 45b, and the resilient hinge 49 connecting the first arm 41 and second arm 42.

The dose divider 30 as described herein may be able to be coupled to multiple types of plunger rod 12, for example plunger rods 12 each having different cross-sections in terms of size and/or shape. Aspects of the dose divider 30 such as the arrangement of the resilient hinge 49 may allow the dose divider 30 to adapt to the particular plunger rod 12 to which it is attached, allowing the opening 58 and channel 47 to expand or contract as necessary so that the plunger rod 12 can be inserted into and then securely held in the channel 47. The cross-sectional profile of the channel 47 may customised such that it matches the cross-sectional profile of a particular plunger rod 12 and/or more than one plunger rod 12. The dose divider 30 as described herein may be adaptable to different shaped plunger rods 12.

Aspects of the present disclosure may be used for intranasal medicament delivery. For example, the medicament 19 contained within the syringe 10 may comprise a Respiratory Syncytial Virus (RSV) vaccine where the required vaccine dose is 0.1 mL per nostril. In this example, the user may fill the container 11 of the syringe 10 with 0.2 mL of the vaccine medicament 19 before coupling a dose divider 30 to the plunger rod 12 of the syringe 10 as described previously. The dose divider 30 in this example should have a dimension L2 that corresponds to a length of the plunger rod 12 that allows for delivery of 0.1 mL of vaccine (i.e. one dose for one nostril). For example, the dose divider may have a dimension L2 between 16 mm and 19 mm, for example a dimension L2 between 17 mm and 18 mm, or more particularly a dimension L2 equal to 17.5 mm. The user administers a 0.1 mL dose of vaccine to the first nostril of a patient before removing the dose divider 30 and administering the remaining 0.1 mL second dose of vaccine to the second nostril of the patient, in a manner as discussed previously. In some examples, the required vaccine dose may be different to 0.1 mL per nostril, while in some examples the medicament 19 may be a different type of vaccine to an RSV vaccine, or a different medicament 19 to a vaccine. In some examples, aspects of the present disclosure may be used for different forms of medicament delivery to intranasal medicament delivery, for example, but not limited to, subcutaneous injection or intramuscular injection.

In some examples, it has been described that the user couples the dose divider 30 to the plunger rod 12 of the syringe 10 prior to medicament administration. However, it should be understood that in some examples the dose divider 30 may already be coupled to the plunger rod 12 before the user receives the syringe 10, e.g. coupled by a third party manufacturer.

In some examples the user performing the medicament administration may fill the container 11 of the syringe 10 with medicament 19, for example prior to coupling the dose divider 30 to the plunger rod 12. However in other examples the container 11 of the syringe 10 may have been pre-filled with medicament 19 before the user receives the syringe 10, e.g. pre-filled by a third party manufacturer.

Described herein are systems, devices, components, and methods that may be associated with delivering a respiratory syncytial virus (RSV) vaccine using an intranasal atomization delivery device.

As used herein, “RSV ΔNS2/Δ1313/I1314L” refers to an RSV ΔNS2/Δ1313/I1314L (NIH) or an RSV ΔNS2/Δ1313/I1314L (Sanofi). Each of the ΔNS2/Δ1313/I1314L (NIH) and the RSV ΔNS2/Δ1313/I1314L (Sanofi) comprise a live-attenuated RSV with (i) a 523 nucleotide (nt) deletion of the NS2 gene (ΔNS2), (ii) an amino acid deletion in the L protein, and (iii) a genetically stabilizing mutation in the L gene. The live-attenuated RSV of the RSV ΔNS2/Δ1313/I1314L (Sanofi) also includes a nucleotide modification at position 14456 that represents a change from a thymine (T) to an adenine (A) in a non-coding region.

As used herein, “RSV ΔNS2/Δ1313/I1314L vaccine” refers to an “RSV ΔNS2/Δ1313/I1314L (NIH) vaccine” or an “RSV ΔNS2/Δ1313/I1314L (Sanofi) vaccine.” An RSV ΔNS2/Δ1313/I1314L (NIH) vaccine comprises an effective amount of RSV ΔNS2/Δ1313/I1314L (NIH). An RSV ΔNS2/Δ1313/I1314L (Sanofi) vaccine comprises an effective amount of RSV ΔNS2/Δ1313/I1314L (Sanofi).

### Exemplary Methods and Uses

In some embodiments, provided are methods of delivering a dose of an RSV vaccine using an intranasal atomization delivery device.

In some embodiments, the methods of administering a dose of an RSV vaccine that comprises a live-attenuated RSV use an intranasal atomization delivery device. In some embodiments, provided are methods of administering a dose of an RSV vaccine using an intranasal atomization delivery device, the RSV vaccine comprising an effective amount of a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/I1314L (NIH) vaccine) or RSVΔ NS2/Δ1313/I1314L (Sanofi) vaccine that further comprises a nucleotide modification in a non-coding region that represents a change from a thymine (T) to an adenine (A). In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (I1314L).

In some embodiments, provided are methods of administering a dose of an RSV vaccine using an intranasal atomization delivery device. In some embodiments, the pediatric subject may be 6 to 18 months of age.

In some embodiments, the RSV vaccine is delivered intranasally using the intranasal atomization delivery device to deliver about ½ dose to each nostril. In some embodiments, the RSV vaccine is delivered intranasally so that the whole dose is delivered to one nostril. In some embodiments, the RSV vaccine is delivered intranasally with ½ dose delivered to one nostril and the other ½ dose is delivered to the same nostril after the first ½ dose is absorbed. In some embodiments, the RSV vaccine is delivered intranasally using the intranasal atomization delivery device to deliver a dose in unequal amounts to one or both nostrils.

In some embodiments, the RSV vaccine is delivered intranasally using the intranasal atomization delivery device in a liquid formulation. In some embodiments, the RSV vaccine dose is delivered intranasally using the intranasal atomization delivery device in about 0.2 mL. In some embodiments, the 0.2 mL dose is delivered intranasally wherein about 0.1 mL is delivered to each nostril. In some embodiments, the RSV vaccine dose is delivered intranasally using the intranasal atomization delivery device in about 0.01 mL, 0.02 mL, 0.05 mL, 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL or 1.5 mL. As described above, the dose may be divided evenly between two nostrils, divided unevenly between two nostrils, or delivered all to one nostril in one or more deliveries.

In some embodiments, provided are methods of administering a first dose of an RSV vaccine using an intranasal atomization delivery device and administering a second dose of the RSV vaccine using an intranasal atomization delivery device.

In some embodiments, provided are methods of administering a first dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, provided are methods of administering a dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, the intranasal atomization delivery device comprises a barrel operably connected to the spray nozzle and a plunger movable within the barrel to advance the RSV vaccine through the spray nozzle. In some embodiments, the intranasal atomization delivery device further includes a dose divider for splitting the dose of the RSV vaccine into two or more deliveries. In some embodiments, the dose divider may divide the dose of the RSV vaccine in about half of a volume to be delivered to a subject. In some embodiments the dose divider is used to deliver ½ dose to each nostril of the subject.

In some embodiments, the intranasal atomization delivery device delivers an average droplet size Dᵥ₅₀ of 10-120 μm. In some embodiments, the intranasal atomization delivery device delivers an average droplet size Dᵥ₅₀ of 10-120 μm, about 30-110 μm, about 50-110 μm, about 70-110 μm, or about 80-110 μm. In some embodiments, the intranasal atomization delivery device delivers an average shot weight between about 95 mg to about 135 mg, between about 100 mg to about 130 mg, or between about 100 mg to about 130 mg or between about 105 mg to about 130. In some embodiments, the intranasal atomization delivery device delivers an average shot volume of about 85 μL to about 120 μL, about 90 μL to about 115 μL, or about 95 μL to about 115 μL.

In some embodiments, use of an intranasal atomization delivery device described herein for administering a dose of an RSV vaccine to a subject is provided wherein the RSV vaccine comprises an effective amount of a live-attenuated RSV. In some embodiments, the use includes a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/I1314L (NIH) vaccine) or RSVΔ NS2/Δ1313/I1314L (Sanofi) vaccine that further comprises a nucleotide modification in a non-coding region that represents a change from a thymine (T) to an adenine (A).

### Immunogenicity

Approximately seventy percent (70%) of vaccine recipients attained a 4-fold response in neutralizing antibody titer following the second vaccine dose in both low and high dose groups, compared to 61 and 47% following one vaccine dose in low and high dose RSV naïve participants. This increase in the percentage of participants attaining a 4-fold response following a second administration supports the use of a second vaccine administration in this population. The 70% attaining this fold rise post second vaccine administration aligns with the expected clinical efficacy goal of 70% for the candidate. After one or two vaccinations, 75% attained a 4-fold rise in serum neutralizing antibody titers. The percentage of RSV experienced participants (36% and 22% in the low and high dose groups respectively) also attaining a 4-fold response in neutralizing antibody titers post vaccination 1 suggests potential benefit for this sub-group as well. It must be noted that this comes from a modest sample of participants (n=20 of 97 vaccine recipients) at this point of interim analysis.

Taken together, these data strongly support the use of RSV ΔNS2/Δ1313/I1314L (Sanofi) vaccine at an operative range study for the candidate including doses at 5.6 and 6.2 log PFU/dose.

### Vaccine Virus Shedding and Infectivity

Following each vaccine administration, the shedding of vaccine virus was considered in addition to the fold rise in neutralizing antibody titers or serum IgG as vaccine infectivity. The high level of vaccine infectivity (over 80% and 70% in RSV naïve following the first and second vaccine administration respectively) is supportive of a promising vaccine candidate associated with good vaccine take. When infectivity was considered after either vaccine administration, over 90% of participants had evidence of infection. In the small cohort of RSV experienced participants, relatively high infectivity (80% and 33.3% in low and high dose recipients following one vaccine administration and 60 and 50% following a second vaccine administration) was found, implicating promise for this group. In addition, the marked drop in the percentage of vaccine virus shedders in RSV naïve participants after the second vaccine administration (roughly 20%) compared to the first vaccine administration (over 70%) is as previously documented with other efficacious live-attenuated mucosal viral vaccines where subsequent ‘challenge’ in the form of a second vaccine dose is characterized by a marked reduction in vaccine virus shedding. Of note, the shedding data available for this cohort was from data at a single timepoint following each vaccination (seven days post vaccination). While this coincides with the point of peak viral shedding documented in other RSV live-attenuated vaccine (LAV) trials, it is likely that some shedders may have been missed. This limitation in the available shedding data makes the results obtained particularly encouraging.

### Overall Conclusions

Interim analysis results showed a promising safety, immunogenicity and infectivity profile of the RSV ΔNS2/Δ1313/I1314L (Sanofi) candidate.

No safety concerns were identified after 1- and 2-dose administrations of either dose level of the investigational RSV ΔNS2/Δ1313/I1314L (Sanofi) vaccine or by baseline serostatus.

The vaccine virus shedding, and immunogenicity conclusions based on the IgA serostatus at baseline show marked vaccine take demonstrated at both dose levels, and 70% of vaccine RSV-naïve recipients attained a 4-fold response in serum neutralizing antibody responses post the second vaccine administration for both dose levels in RSV-naïve participants.

### REFERENCE NUMERAL KEY

10 syringe
11 medicament container
12 plunger rod
13 finger grip
14 outer side wall of container
15 medicament chamber
16 outlet
17 atomizer
18 piston
19 medicament
20 end surface
21 distal face
30 dose divider
41 first arm
42 second arm
44a front portion of first arm
44b front portion of second arm
45a rear portion of first arm
45b rear portion of second arm
46a inner surface of front portion of first arm
46b inner surface of front portion of second arm
47 channel
48a inner surface of rear portion of first arm
48b inner surface of rear portion of second arm
49 resilient hinge
50a first straight portion
50b second straight portion
51 curved portion
52a, 52b grip formation
53a outer surface of rear portion of first arm
53b outer surface of rear portion of second arm
54a-f, 54a′-f′ ridges
55 top surface
56 bottom surface
57a, 57b lip
58 opening
60 first guide surface
61 second guide surface
62 blocking feature
63 first blocking projection
64 second blocking projection
66 stop formation
67 first protrusion
68 second protrusion
69 groove
70 medicament delivery device

1. A dose divider (30) for releasable attachment to a plunger rod (12) of a syringe (10), the dose divider comprising:
a first arm (41) and a second arm (42) opposite the first arm, each comprising a respective front portion (44a, 44b), wherein the front portion (44a) of the first arm and the front portion (44b) of the second arm form a channel (47) therebetween for receiving the plunger rod; and
a resilient hinge (49) pivotably coupling the first arm and the second arm such that the dose divider is reversibly movable between a first configuration and a second configuration;
wherein when the dose divider is in the second configuration, the dose divider is configured to receive the plunger rod in the channel through an opening formed between the front portion of the first arm and the front portion of the second arm, the channel having a shape configured to receive a specific outer shape of the plunger rod; and
wherein the front portion of the first arm and the front portion of the second arm are closer when the dose divider is in the first configuration than when the dose divider is in the second configuration.

2. A dose divider according to claim 1, wherein the resilient hinge is configured to bias the dose divider towards the first configuration.

3. A dose divider according to claim 1 or 2, wherein the first arm (41) and the second arm (42) each comprises a respective rear portion (45a, 45b);
wherein the dose divider is configured to be moved from the first configuration to the second configuration by a user moving the rear portion (45a) of the first arm towards the rear portion (45b) of the second arm.

4. A dose divider according to claim 3, wherein an outer surface of the rear portion of the first arm and an outer surface of the rear portion of the second arm comprise respective grip formations (52a, 52b), optionally wherein each grip formation comprises a plurality of ridges (54a-f, 54a′-f′).

5. A dose divider according to claim 3 or 4, comprising a stop formation (66) located on at least one of the rear portion of the first arm or the rear portion of the second arm;
wherein the stop formation is located between the first arm and the second arm to limit movement of the rear portion of the first arm towards the rear portion of the second arm.

6. A dose divider according to claim 5, wherein the stop formation comprises a first protrusion (67) located on the rear portion of the first arm and a second protrusion (68) located on the rear portion of the second arm;
wherein the first protrusion and the second protrusion are configured to be brought into contact with each other as the rear portion of the first arm is moved towards the rear portion of the second arm to limit further movement of the rear portion of the first arm towards the rear portion of the second arm.

7. A dose divider according to claim 6, wherein the first protrusion comprises a groove (69) configured to receive at least a portion of the second protrusion when the first protrusion and the second protrusion are brought into contact with each other.

8. A dose divider according to any one of the preceding claims, wherein the front portion of the first arm and the front portion of the second arm each comprises a respective guide surface (60, 61) for guiding the plunger rod through the opening towards the channel.

9. A dose divider according to any one of the preceding claims, comprising at least one of a first blocking projection (63) or a second blocking projection (64) located between the channel and the resilient hinge to inhibit movement of the plunger rod out of the channel.

10. A dose divider according to any one of the preceding claims, wherein the dose divider is integrally formed from a single piece of material, optionally wherein the material is a polymer.

11. A dose divider according to any one of the preceding claims, wherein the resilient hinge comprises a curved portion (51) coupled at either end to a respective straight portion (50a, 50b).

12. A dose divider according to claim 11, wherein the curved portion curves away from a front end of the dose divider such that a vertex of the curved portion is at a rear of the resilient hinge.

13. A dose divider according to any one of claims 1 to 10, wherein the resilient hinge is arcuate.

14. A dose divider according to any one of the preceding claims, wherein the resilient hinge is a living hinge integrally formed with the first arm and the second arm.

15. A medicament delivery device (70) comprising a dose divider (30) according to any preceding claim and a syringe (10), wherein the syringe comprises the plunger rod (12) and the dose divider is removably coupled to the plunger rod.

16. A medicament delivery device according to claim 15, wherein the syringe comprises an atomizer (17) and is configured for intranasal delivery of a medicament.

17. A medicament delivery device according to claim 16, wherein the medicament (19) is an RSV vaccine.

18. A system comprising:
a vial containing a medicament;
a syringe (10) comprising a container (11) and a plunger rod (12);
a needle configured to be coupled to the syringe such that the medicament can be drawn via the needle from the vial into the container (11);
an atomizer (17) configured to be coupled to the syringe; and
a dose divider (30) according to any of claims 1 to 14 configured to be coupled to the plunger rod (12).

19. A system according to claim 18, wherein the medicament comprises a vaccine, optionally wherein the vaccine is an RSV vaccine.

20. A system according to claim 18 or 19, wherein the syringe is configured for intranasal delivery of the medicament.

21. A medicament delivery device (70) comprising:
a dose divider (30) comprising:
a first arm (41) and a second arm (42) opposite the first arm, each comprising a respective front portion (44a, 44b);
a resilient hinge (49) pivotably coupling the first arm and the second arm such that the dose divider is reversibly movable between a first configuration and a second configuration; and
a syringe (10) comprising a plunger rod (12) having an outer shape;
wherein the front portion (44a) of the first arm and the front portion (44b) of the second arm form a channel (47) therebetween forming a shape configured to receive the outer shape of the plunger rod; and
wherein the dose divider is removably coupled to the plunger rod.

22. A medicament delivery device according to claim 21, wherein the syringe comprises an atomizer (17) and is configured for intranasal delivery of a medicament.

23. A medicament delivery device according to claim 22, wherein the medicament (19) is an RSV vaccine.

24. A system comprising:
a vial containing a medicament;
a syringe (10) comprising a container (11) and a plunger rod (12);
a needle configured to be coupled to the syringe such that the medicament can be drawn via the needle from the vial into the container (11);
an atomizer (17) configured to be coupled to the syringe; and
a dose divider (30) configured to be coupled to the plunger rod (12), the dose divider comprising a first arm (41) and a second arm (42) opposite the first arm, and a resilient hinge (49) pivotably coupling the first arm and the second arm such that the dose divider is reversibly movable between a first configuration and a second configuration.

25. A system according to claim 24, wherein the medicament comprises a vaccine, optionally wherein the vaccine is an RSV vaccine.

26. A system according to claim 24 or 25, wherein the syringe is configured for intranasal delivery of the medicament.
